(19) United States
(12) Patent Application Publication
(10) Pub. No.: US 2007/0198735 A1
(43) Pub. Date: Aug. 23, 2007

(54) Method and system for supporting RSVP in IPv4/IPv6 hybrid network

(76) Inventors: Bong-Chan Kim, Seoul (KR); Sun-Gi Kim, Seoul (KR); Jae-Hwoon Lee, Seoul (KR); Kang-Young Moon, Yongin-si (KR)

Correspondence Address: Robert E. Bushnell, Suite 300, 1522 K Street, N.W., Washington, DC, 20005, US

(21) Appl. No.: 11/649,158

(22) Filed: Jan. 4, 2007

(30) Foreign Application Priority Data
Feb. 17, 2006 (KR) 10-2006-0015844

(51) Int. Cl. G06F 15/16

(52) U.S. Cl. 709/230

(57) ABSTRACT

In a method and system for supporting resource reservation protocol (RSVP) in an Internet protocol version 4 (IPv4)/Internet protocol version 6 (IPv6) hybrid network, the method includes the steps of: transmitting, from a dual stack host in an IPv6 network, an end-to-end quality of service (QoS) session establishment request message to an IPv4 server through a dual stack transition mechanism tunnel end point (DSTM TEP); transmitting, from the IPv4 server, an end-to-end path message to the dual stack host through the DSTM TEP; transmitting, from the DSTM TEP to the dual stack host, a path message for reserving resources in the IPv6 network; transmitting, from the dual stack host, an end-to-end resource reservation request message to the IPv4 server through the DSTM TEP, and making a resource reservation in an IPv4 network; and transmitting, from the dual stack host to the DSTM TEP, a resource reservation request message, and making a resource reservation in the IPv6 network.

### CLAIM OF PRIORITY

This application makes reference to, incorporates the same herein, and claims all benefits accruing under 35 U.S.C. §119 from an application for RESOURCE RESERVATION PROTOCOL SUPPORTING SYSTEMAND METHOD ININTEGRATED IPv4/IPv6 NETWORK earlier filed in the Korean Intellectual Property Office on the 17^{th} of Feb. 2006 and there duly assigned Serial No. 10-2006-0015844.

### BACKGROUND OF THE INVENTION

1. Technical Field

The present invention relates to a method and system for supporting resource reservation protocol (RSVP) in an Internet protocol version 4 (IPv4)/Internet protocol version 6 (IPv6) hybrid network.

2. Related Art

The Internet has become the core infrastructure of the twenty-first century information oriented society. Traffic transmitted through the Internet has changed from text traffic of the past into multimedia traffic including sound, image and video, and high quality multimedia traffic requiring real-time processing, such as voice over Internet protocol (VoIP) service and Internet television service, is explosively increasing.

Multimedia traffic carries a tremendous amount of data compared to conventional text traffic, and is sensitive to delay and jitter. Therefore, efforts have been made to provide quality of service (QoS) required for traffic, such as voice, video and data, of triple play service (TPS) having different characteristics in the current Internet.

With the increase in such multimedia traffic and generalization of Internet use, the amount of traffic transmitted through the Internet is doubling every six months, and this tendency is predicted to continue in the future. Thus, it is necessary to build Internet infrastructure capable of efficiently accepting the rapid increase in Internet traffic.

The current Internet is based on IPv4. According to IPv4, a source records a source address and a destination address in a packet and transmits the packet through the Internet in order to transmit traffic to the destination. Roughly, the Internet may be regarded as a router-based network in which, when a router receives the packet, it determines an interface to transmit the packet using its own routing table.

The router compares the length of the packet with a maximum transmission unit (MTU) of the interface, and when the packet length is larger than the MTU, the router fragments the packet. The router determines whether the packet has an option and performs a proper action. However, the fragmentation, option checking and processing deteriorate performance of the router, and thus overall performance of the Internet.

Furthermore, an IPv4 address has 32 bits, enabling a maximum of approximately four billion hosts to have access to the Internet. A very small number of hosts, however, are actually allowed to access the Internet due to the use of special addresses, subnetting, and inefficient address allocation caused by network address allocation.

Meanwhile, with the generalization of the Internet and the increase in multimedia traffic, efforts have been continuously made to connect mobile communication terminals, electronic information appliances and the like, as well as computers, to the current Internet, unlike in the past. There are a great number of electronic information appliances, such as mobile communication terminals, televisions and refrigerators, and IPv4 addresses are extremely insufficient to connect such appliances to the Internet.

Therefore, IPv6 technology has been suggested because it allows a substantially unlimited number of terminals to connect to the Internet and improves the overall performance of the Internet by addressing the inefficiency of IPv4.

IPv6 is a protocol capable of preventing performance deterioration of the Internet caused by drawbacks of IPv4, and capable of efficiently accepting multimedia traffic needing real-time transmission, the drawbacks of IPv4 being caused because all transit routers between a source and a destination should check all options and perform fragmentation.

In addition, IPv6 uses a 128-bit address system, and thus it can accommodate a substantially unlimited number of terminals. When an address system is changed from 32 bits to 128 bits, the content of a routing table required for a router to determine a path may increase. Since the address system of IPv6 has a structure of more layers than that of IPv4, it does not take a long time to find a proper path from the routing table.

That is, the improved functions of IPv6 can resolve the performance problem of the current Internet caused by the rapid increase in Internet traffic and generalization of multimedia traffic.

While IPv6 has considerably improved functions over IPv4, the current Internet is based on IPv4, and IPv4 and IPv6 are not directly compatible with each other. In addition, it is impossible to quickly change IP functions of all routers and hosts constituting the current Internet.

Therefore, a shift from IPv4 to IPv6 will be gradually realized over a considerably long time. The Internet will be based upon IPv4 at the time when IPv6 is initially introduced, and then an IPv6 network will expand in size from utilization at specific enterprises or research institutes while the IPv4 network will reduce, achieving a natural shift to IPv6. Thus, it will be a main factor of the success of IPv6 to provide a mechanism capable of transmitting information between an IPv6 network connected host and an IPv4 network connected host.

Accordingly, a transition mechanism is necessary to provide mutual compatibility, allowing hosts located in an IPv6 network and an IPv4 network to transmit and receive traffic between each other.

For such a transition mechanism, the Internet Engineering Task Force (IETF) suggested a translation mechanism such as network address translation-protocol translation (NAT-PT) and a tunneling mechanism such as dual stack transition mechanism (DSTM). NAT-PT combines stateless IP/Internet control message protocol translator (SIIT) protocol translation and proper dynamic address translation of application level gateway (ALG), such as NAT and a domain name system (DNS) ALG, thereby allowing mutual communication between an IPv6-only node and an IPv4-only node.

In other words, NAT-PT allows an IPv4 address to be dynamically allocated using an IPv4 address pool at a boundary between an IPv6 network and an IPv4 network. The NAT-PT binds an IPv6 network address to an IPv4 network address, and provides datagrams transmitted and received between address areas with transparent routing.

In other words, nothing is changed at a terminal node using the NAT-PT mechanism, and IP packet routing becomes completely transparent to the terminal node. However, there is a restriction on the application of the NAT-PT transition mechanism. All responses and requests for one session should be routed through the same NAT-PT router.

Furthermore, there is a difference in meanings between IPv4 fields and IPv6 fields, not allowing direct translation from IPv4 to IPv6. In addition, since the NAT-PT performs address translation in an IP layer, an application using an IP address in an upper layer does not normally work. An ALG is used to solve the problem. It is, however, essentially impossible to make an ALG in consideration of all of currently used applications and newly developed applications. Therefore, the address translation related problems cannot be resolved.

Furthermore, the most important problem associated with NAT-PT is that security of an end-to-end network layer is impossible. When an IP address is used in the application layer, security of transport and application layers is also impossible. This is a limitation of the NAT function itself. Meanwhile, since IP security protocol (IPSec) security is impossible between different address areas, end nodes that want IPSec network level security should both support IPv4 or IPv6. In addition, IPSec security has a drawback in that domain name system (DNS)-security (SEC) cannot be applied.

On the other hand, terminals located in an IPv6 network implement two protocol stacks of IPv4 and IPv6, and DSTM enables each terminal to communicate using the IPv6 stack when it connects to an IPv6 node, and enables the terminal to communicate using the IPv4 stack according to an IPv4-in-IPv6 tunneling mechanism when it connects to an IPv4 node.

The DSTM provides a method for providing a temporary global IPv4 address to an IPv6 node, and for transmitting an IPv4 packet using a dynamic tunnel in an IPv6 network. The DSTM also provides a series of processes and architecture defining essential support infrastructure for this transition mechanism.

When necessary, the DSTM designates an IPv4 address to a dual IP layer host. Then, an IPv6 host can communicate with an IPv4-only host, or an IPv4-only application can be executed without being modified at the IPv6 host.

In other words, the DSTM is composed of a DSTM server, a tunnel end point (TEP), and a DSTM client, and when the DSTM client attempts to connect to an IPv4 node in an IPv4 network, it is allocated a temporary global IPv4 address by the DSTM server.

When the DSTM client generates an IPv4 packet using the information in order to communicate with the IPv4 node, the node encapsulates the IPv4 packet using its own IPv6 address and IPv6 address information of the TEP, and then transmits the packet. The packet is an IPv6 packet. Thus, in a network including a DSTM node, the packet is transmitted to the TEP by means of an internal routing algorithm.

When receiving the packet, the TEP builds a mapping table using the address included in an IPv6 header and the source IPv4 address of the IPv4 packet included in a payload of the IPv6 packet, removes the IPv6 header, and then transmits the IPv4 packet to the IPv4 node. The IPv4 node then transmits a reply packet in response to the received traffic, and the TEP receives the packet and sends it to the IPv6 node using tunneling.

Meanwhile, RSVP is defined, RSVP being a resource reservation scheme to accommodate traffic requiring different QoS in an IP layer. According to the RSVP method, network resources are previously reserved for connection based on QoS, and when the reservation is not possible, resource reservation is rejected.

The RSVP method is a technique which reserves required resources of all routers between a source and a destination in order to establish a flow, which is a type of virtual line, from the source to the destination, thereby ensuring QoS. The RSVP method may be called a flow-based model.

In other words, RSVP may be defined as a signaling protocol for ensuring QoS based on a flow, and routers between a source node and a destination node reserve their resources using the signaling protocol.

When a network to which the source node belongs is an IPv6 network, and a network to which the destination node belongs is an IPv4 network, interworking is essential between an RSVP operating in the IPv6 network and an RSVP operating in the IPv4 network in order to ensure QoS between the IPv6 network and the IPv4 network. However, only resource reservation in a single network, such as an IPv4-based network or an IPv6-based network, is defined in current RSVP.

As described above, a conventional RSVP supporting mechanism allows establishment of a session for providing QoS in only a single network, such as an IPv4 network or an IPv6 network, but it cannot allow a session for providing QoS to be established in an IPv6-IPv4 hybrid network environment in which an IPv6 network and an IPv4 network are combined.

In addition, because the current Internet is based on IPv4, servers such as a streaming server storing and transmitting traffic requiring real-time processing are generally located in an IPv4 network. Therefore, there is no series of processes for a dual stack host in an IPv6 network to establish a QoS session with a server located in the IPv4 network.

### SUMMARY OF THE INVENTION

It is an object of the present invention to provide a method and system for supporting resource reservation protocol (RSVP) in an Internet protocol version 4 (IPv4)/IPv6 hybrid network in which IPv4 and IPv6 networks coexist, wherein when a server for multimedia traffic is located in an IPv4 network, an end-to-end RSVP connection is established so that traffic transmitted from the server to a dual stack host located in an IPv6 network can be assured quality of service (QoS).

According to an aspect of the present invention, a method for supporting RSVP in an IPv4/IPv6 hybrid network comprises the steps of: transmitting, from a dual stack host in an IPv6 network, an end-to-end QoS session establishment request message to an IPv4 server through a dual stack transition mechanism tunnel end point (DSTM TEP); transmitting, from the IPv4 server, an end-to-end path message to the dual stack host through the DSTM TEP; transmitting, from the DSTM TEP, a path message for reserving resources in the IPv6 network to the dual stack host; transmitting, from the dual stack host, an end-to-end resource reservation request message to the IPv4 server through the DSTM TEP, and making a resource reservation in an IPv4 network; and transmitting, from the dual stack host, a resource reservation request message to the DSTM TEP, and making a resource reservation in the IPv6 network.

In the step of transmitting, from a dual stack host in an IPv6 network, an end-to-end QoS session establishment request message to an IPv4 server through a DSTM TEP, the QOS session establishment request message may have an IPv4 header, and may be encapsulated in an IPv6 packet and transmitted to the DSTM TEP by IPv4-in-IPv6 tunneling.

The DSTM TEP stores, in a table, mapping information between a source IPv6 address included in the message transmitted from the dual stack host and an internal source IPv4 address, and then transmits an IPv4 packet from which an IPv6 header is removed to the IPv4 server.

In the step of transmitting, from the IPv4 server, an end-to-end path message to the dual stack host through the DSTM TEP, the path message transmitted to the DSTM TEP may have a structure which includes path data, an RSVP header, and an IPv4 header.

A destination address of an IPv4 packet, including the end-to-end path message, is an IPv4 address of the dual stack host.

The DSTM TEP extracts an IPv6 address, mapped to an IPv4 address corresponding to the destination address of the IPv4 packet, from a mapping table, encapsulates the IPv6 address in an IPv6 packet, and then transmits the IPv6 packet to the dual stack host using IPv4-in-IPv6 tunneling.

The path message transmitted by IPv4-in-IPv6 tunneling may have a structure including path data, an RSVP header, an IPv4 header, and an IPv6 header.

In the step of transmitting, from the DSTM TEP, a path message for reserving resources in the IPv6 network to the dual stack host, the path message may have a structure including path data, an RSVP header, and an IPv6 header.

In the step of transmitting, from the dual stack host, an end-to-end resource reservation request message to the IPv4 server through the DSTM TEP and making a resource reservation in an IPv4 network, the end-to-end resource reservation request message may have an IPv4 header, and may be encapsulated in an IPv6 packet and transmitted to the DSTM TEP by IPv4-in-IPv6 tunneling.

The resource reservation request message transmitted to the DSTM TEP may have a structure including resource reservation request data, an RSVP header, an IPv4 header, and an IPv6 header.

The DSTM TEP may transmit, to the IPv4 server, an IPv4 packet which is the resource reservation request message from which an IPv6 header is removed, the resource reservation request message being transmitted from a dual stack host.

The IPv4 packet transmitted to the IPv4 server may make a resource reservation in each router while being transmitted in a hop-by-hop manner.

In the step of transmitting, from the dual stack host, a resource reservation request message to the DSTM TEP and making a resource reservation in the IPv6 network, the resource reservation request message may have a structure including path data, an RSVP header, and an IPv6 header.

The resource reservation request message transmitted to the DSTM TEP may make a resource reservation in each router while being transmitted in the hop-by-hop manner.

According to another aspect of the present invention, a system for supporting RSVP in an IPv4/IPv6 hybrid network comprises a dual stack host which, when receiving an end-to-end path message transmitted from an IPv4 server through a DSTM TEP, transmits an end-to-end resource reservation request message to the IPv4 server through the DSTM TEP making a resource reservation in the IPv4 network, and transmits a resource reservation request message to the DSTM TEP making a resource reservation in the IPv6 network.

The end-to-end path message transmitted to the DSTM TEP may have a structure including path data, an RSVP header, and an IPv4 header.

A destination address of an IPv4 packet, including the end-to-end path message, may be an IPv4 address of the dual stack host.

The DSTM TEP extracts an IPv6 address mapped to an IPv4 address corresponding to the destination address of the IPv4 packet from a mapping table, encapsulates the IPv6 address in an IPv6 packet, and then transmits the IPv6 packet to the dual stack host using IPv4-in-IPv6 tunneling.

The path message transmitted by IPv4-in-IPv6 tunneling may have a structure including path data, an RSVP header, an IPv4 header, and an IPv6 header.

The DSTM TEP transmits a path message for reserving resources in the IPv6 network to the dual stack host.

The path message for reserving resources in the IPv6 network may have a structure including path data, an RSVP header, and an IPv6 header.

The end-to-end resource reservation request message transmitted to the DSTM TEP may have an IPv4 header, and may be encapsulated in an IPv6 packet and transmitted to the DSTM TEP by IPv4-in-IPv6 tunneling.

The end-to-end resource reservation request message transmitted to the DSTM TEP may have a structure including resource reservation request data, an RSVP header, an IPv4 header, and an IPv6 header.

The DSTM TEP transmits, to the IPv4 server, an IPv4 packet which is the resource reservation request message from which an IPv6 header is removed, the resource reservation request message being transmitted from the dual stack host.

The IPv4 packet transmitted to the IPv4 server makes a resource reservation in each router while being transmitted in a hop-by-hop manner.

The resource reservation request message transmitted to the DSTM TEP may have a structure including path data, an RSVP header, and an IPv6 header.

The resource reservation request message transmitted to the DSTM TEP makes a resource reservation in each router while being transmitted in the hop-by-hop manner.

### BRIEF DESCRIPTION OF THE DRAWINGS

A more complete appreciation of the invention, and many of the attendant advantages thereof, will be readily apparent as the same becomes better understood by reference to the following detailed description when considered in conjunction with the accompanying drawings in which like reference symbols indicate the same or similar components, wherein:

FIG. 1 is a diagram illustrating a “SESSION_ASSOC” object of a typical resource reservation protocol (RSVP);

FIG. 2 is a diagram illustrating a “NODE_CHAR” object of a typical RSVP;

FIG. 3 is a diagram illustrating the flow of a path message of RSVP in a typical IPv4/IPv6 hybrid network;

FIG. 4 is a diagram illustrating the flow of a reservation message of RSVP in a typical IPv4/IPv6 hybrid network;

FIG. 5 is a diagram illustrating the flow of a tunnel path message of RSVP in a typical IPv4/IPv6 hybrid network;

FIG. 6 is a diagram illustrating the flow of a tunnel reservation message of RSVP in a typical IPv4/IPv6 hybrid network;

FIG. 7 is a diagram illustrating packet transmission through a plurality of tunnels in a typical IPv4/IPv6 hybrid network;

FIG. 8 is a diagram illustrating the configuration of a system for supporting RSVP in an IPv4/IPv6 hybrid network according to an exemplary embodiment of the present invention;

FIG. 9 is a diagram illustrating a method for supporting RSVP in an IPv4/IPv6 hybrid network according to an exemplary embodiment of the present invention;

FIG. 10 is a diagram illustrating a process of transmitting an end-to-end path message in an IPv4/IPv6 hybrid network according to an exemplary embodiment of the present invention;

FIG. 11 is a diagram illustrating a process of transmitting a message for reserving resources in an IPv6 network of an IPv4/IPv6 hybrid network according to an exemplary embodiment of the present invention;

FIG. 12 is a diagram illustrating a process of reserving resources in an IPv4 network of an IPv4/IPv6 hybrid network according to an exemplary embodiment of the present invention; and

FIG. 13 is a diagram illustrating a process of reserving resources in an IPv6 network of an IPv4/IPv6 hybrid network according to an exemplary embodiment of the present invention.

### DETAILED DESCRIPTION OF THE INVENTION

Hereinafter, exemplary embodiments of the present invention will be described in detail with reference to the accompanying drawings. Like elements are denoted by like reference numerals throughout the drawings. In the following description, a detailed description of known functions and configurations incorporated herein has been omitted for conciseness.

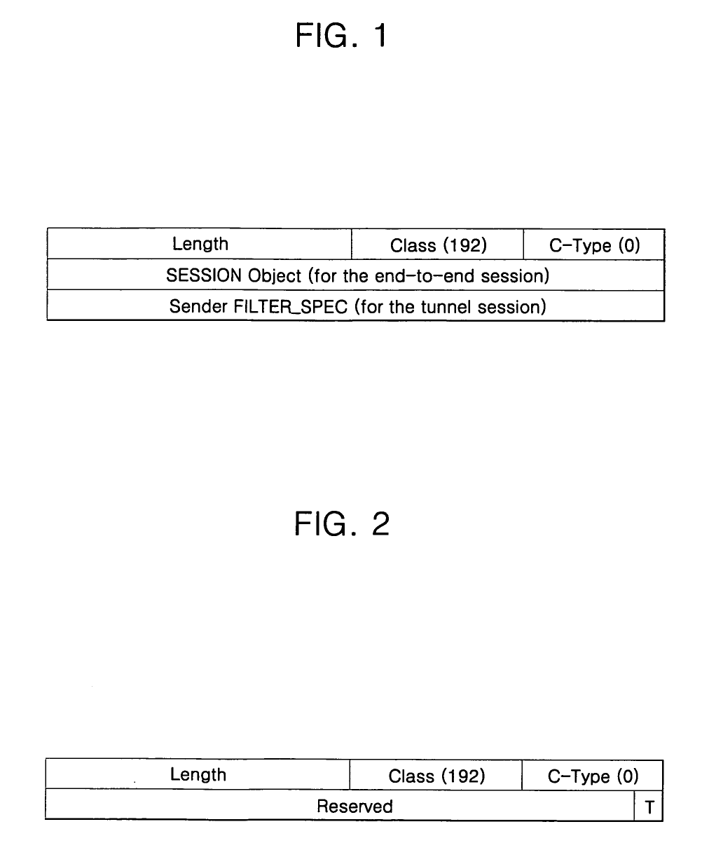

FIG. 1 is a diagram illustrating a “SESSION_ASSOC” object of a typical resource reservation protocol (RSVP).

As illustrated in FIG. 1, the “SESSION_ASSOC” object includes a “Length” field having length information, a “Class” field having class information, a “Type” field having type information, a “SESSION Object” field having end-to-end session information, and a “FILTER_SPEC information” field having tunnel session information.

The “SESSION_ASSOC” object is included in a path message transmitted from a transmitting end to a receiving end.

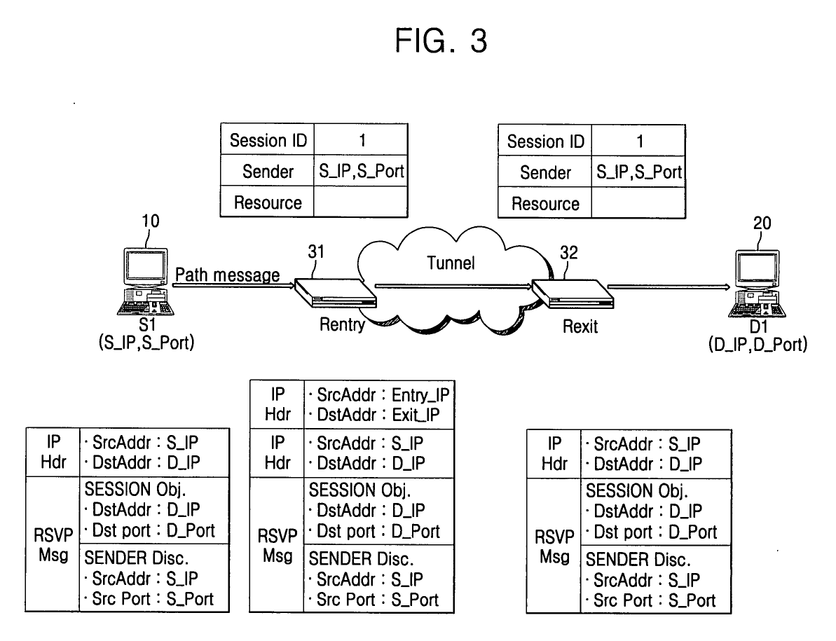

FIG. 2 is a diagram illustrating a “NODE_CHAR” object of a typical RSVP.

A “NODE_CHAR” object is used to enable a final node in a tunnel section so as to deliver, to an initial node in the tunnel section, information as to whether the final node can support a tunnel RSVP mechanism defined by “Request For Comments (RFC) 2746.”

As illustrated in FIG. 2, when a final node in a tunnel section can support the tunnel RSVP mechanism, a “T” bit indicating whether the final node can support the RSVP is set in a “NODE_CHAR” object of a Resv message, which is transmitted from a receiving end to a transmitting end.

FIG. 3 is a diagram illustrating the flow of a path message of RSVP in a typical IPv4/IPv6 hybrid network.

In FIG. 3, a transmitting end (S1) 10 and a receiving end (D1) 20 are in an IPv4 network while a tunnel established between an initial node Rentry 31 and a final node Rexit 32 are in an IPv6 network.

It is assumed that routers and nodes on the paths between the transmitting end 10 and the initial node 31, between the initial node 31 and the final node 32, and between the final node and the receiving end 20 are omitted.

For example, when the transmitting end 10 wants to transmit traffic to the receiving end 20 at 10 Mbps, the transmitting end 10 transmits an end-to-end path message according to RSVP to the receiving end 20 in order to reserve a bandwidth of 10 Mbps between the two ends.

The transmitting end 10 sets source address information of an IP header to its own IP address information “S_IP” and destination address information to IP address information “D_IP” of the receiving end 20.

The transmitting end 10 also sets the destination address information “D_IP” and destination port information “D_Port” in a “SESSION” object of the end-to-end path message, and sets source address information of a “SENDER_TEMPLATE” object to its own address information “S_IP” and source port information to its own port information “S_Port.”

The end-to-end path message is transmitted with a “Router Alert IP option,” and thus is processed by all routers supporting RSVP between the transmitting end 10 and the receiving end 20.

When receiving the path message, the initial node Rentry 31 of the tunnel cannot recognize whether the final node Rexit 32 of the tunnel can support the RSVP mechanism, and thus stores a path status of an end-to-end session, and encapsulates and transmits the path message to Rexit 32.

Rexit 32 decapsulates the received encapsulated path message, sets up a path for the end-to-end session, and then transmits the path message to the receiving end 20.

When receiving the path message, the receiving end 20 transmits a Resv message to the transmitting end 10 in a hop-by-hop manner.

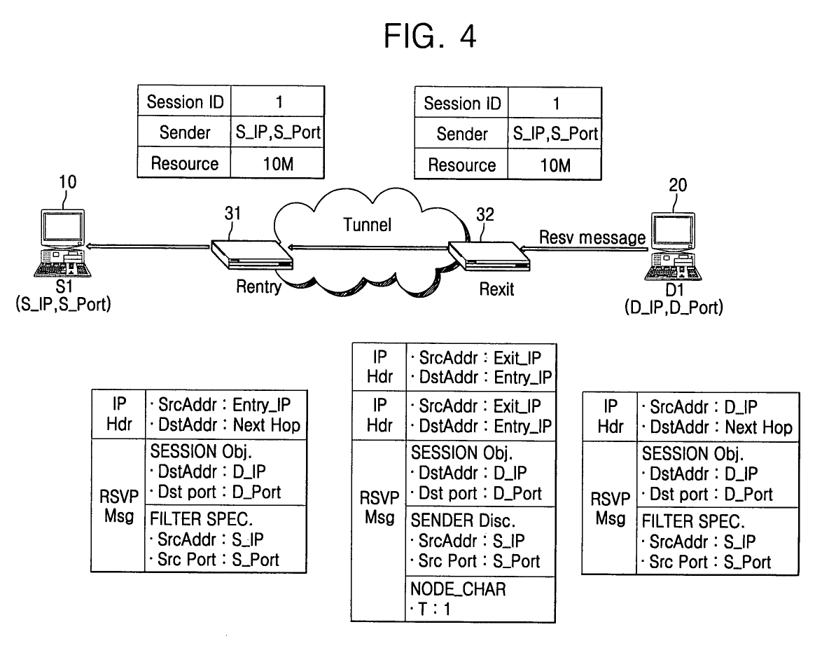

FIG. 4 is a diagram illustrating the flow of a reservation message of RSVP in a typical IPv4/IPv6 hybrid network.

Referring to FIG. 4, the receiving end 20 sets source address information of an IP header (Hdr) to “D_IP” and destination address information to IP address information of Rexit 32, which is an upstream node (Next_Hop) supporting the RSVP mechanism, and transmits to Rexit 32 an RSVP message having a “SESSION” object including the same information as in the path message and a “FILTER SPEC” object including the same information as in a “SENDER_TEMPLATE” object of the path message.

When receiving the Resv message from the receiving end 20, Rexit 32 reserves a resource for an end-to-end session. Since there is no tunnel session mapped to the end-to-end session, Rexit 32 adds to the Resv message a “NODE_CHAR” object having a “T” bit set to inform Rentry 31 that a tunnel RSVP can be supported, and then encapsulates and transmits the Resv message to Rentry 31.

Rentry 31 decapsulates the received Resv message, removes the “NODE_CHAR” object, and then reserves resources for the end-to-end session. In addition, Rentry 31 transmits the Resv message from which the “NODE_CHAR” object is removed to the transmitting end 10.

In this regard, when Rentry 31 can support the tunnel RSVP mechanism, Rentry 31 transmits a tunnel path message to Rexit 32 while transmitting the Resv message to the transmitting end 10 through an uplink.

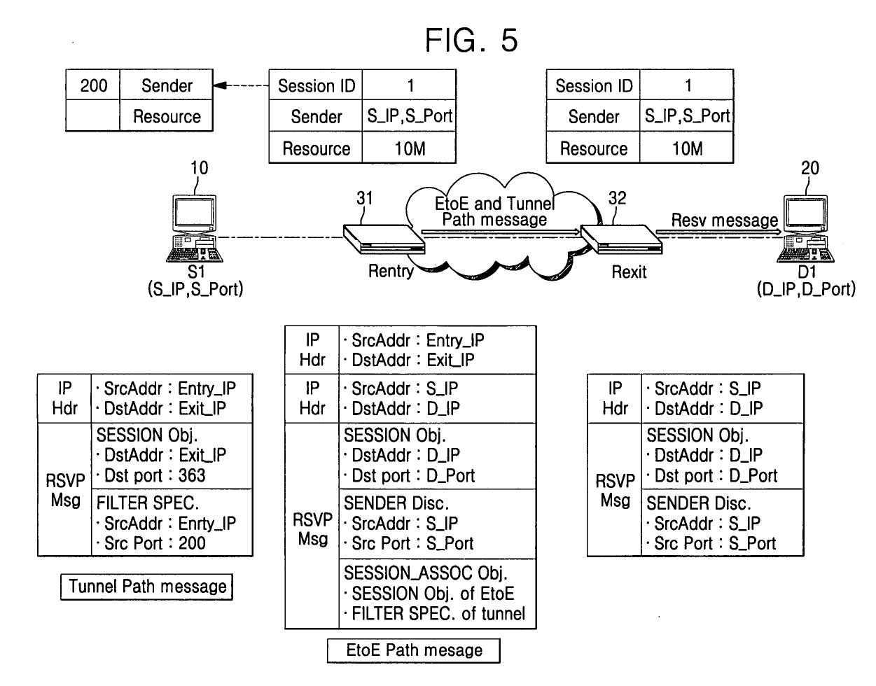

FIG. 5 is a diagram illustrating the flow of a tunnel path message of RSVP in a typical IPv4/IPv6 hybrid network.

Referring to FIG. 5, when receiving a Resv message, Rentry 31 which is an initial node of a tunnel establishes a tunnel session mapped to an end-to-end session, and transmits a tunnel path message and an end-to-end path message for indicating changed information on a path to Rexit 32 in order to reserve resources in the tunnel.

Since the transmitting node of the tunnel path message is Rentry 31 and the receiving node is Rexit 32, source address information of an IP header is set to “Entry_IP” which is address information of Rentry 31, destination address information is set to “Exit_IP,” destination address information of a “SESSION” object is set to “Exit_IP”, and destination port information is set to, for example, “363.”

Additionally, in a “SENDER_TEMPLATE” object of the tunnel path message, source address information becomes “Entry_IP,” and source port information becomes a specific value allocated by Rentry 31 in order to identify each flow in the tunnel.

Such a tunnel path message allows nodes which exist in the tunnel established between Rentry 31 and Rexit 32, and which support RSVP, to set up a path for the tunnel session.

In addition, the end-to-end path message, including a “SESSION_ASSOC” object indicating mapping information between the end-to-end session and the tunnel session, allows Rexit 32 to map the tunnel session and the end-to-end session.

When receiving the end-to-end path message, Rexit 32 sets up mapping information corresponding to the “SESSION_ASSOC” object of the end-to-end path message, removes the “SESSION_ASSOC” object, and then transmits a tunnel Resv message to Rentry 31, in order to request resources of the tunnel session mapped in the end-to-end session, along the path set toward the receiving end in the tunnel.

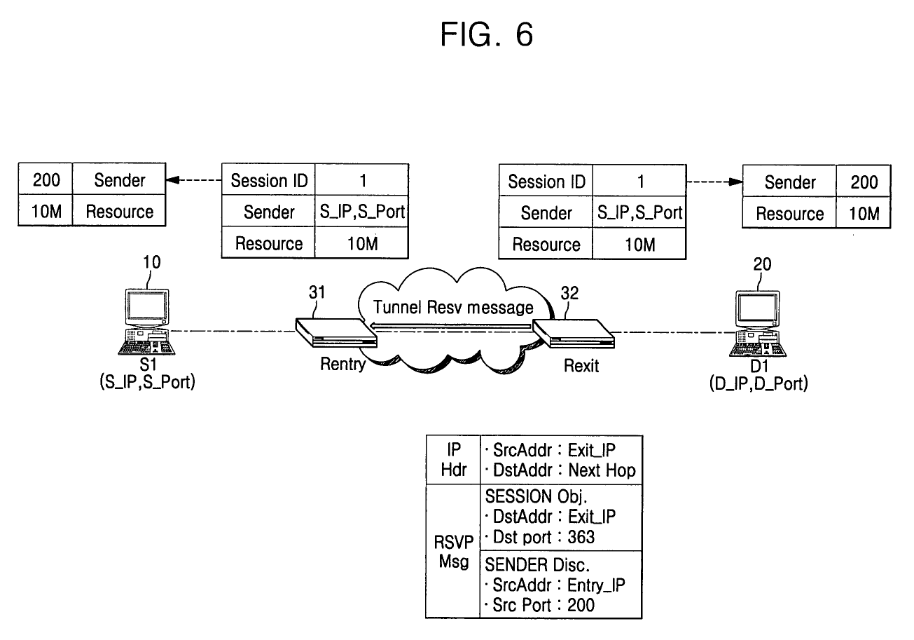

FIG. 6 is a diagram illustrating the flow of a tunnel reservation message of RSVP in a typical IPv4/IPv6 hybrid network.

Referring to FIG. 6, source address information included in an IP header of a tunnel Resv message is set to “Exit_IP,” and destination address information is set to address information of an upstream node (Next_Hop) of Rexit 32 supporting RSVP.

In addition, in a “SESSION” object of the tunnel Resv message, destination address information is set to “Exit_IP” and destination port information is set to “363.” In a “FILTER SPEC” object, source address information is set to “Entry_IP” and source port information is set to a value (e.g., 200) allocated for a tunnel session corresponding to an end-to-end session by Rentry 31.

The tunnel Resv message is transmitted to RSVP-supportable nodes in the tunnel which is established between Rexit 32 and Rentry 31 in the hop-by-hop manner, allowing each node to reserve tunnel resources.

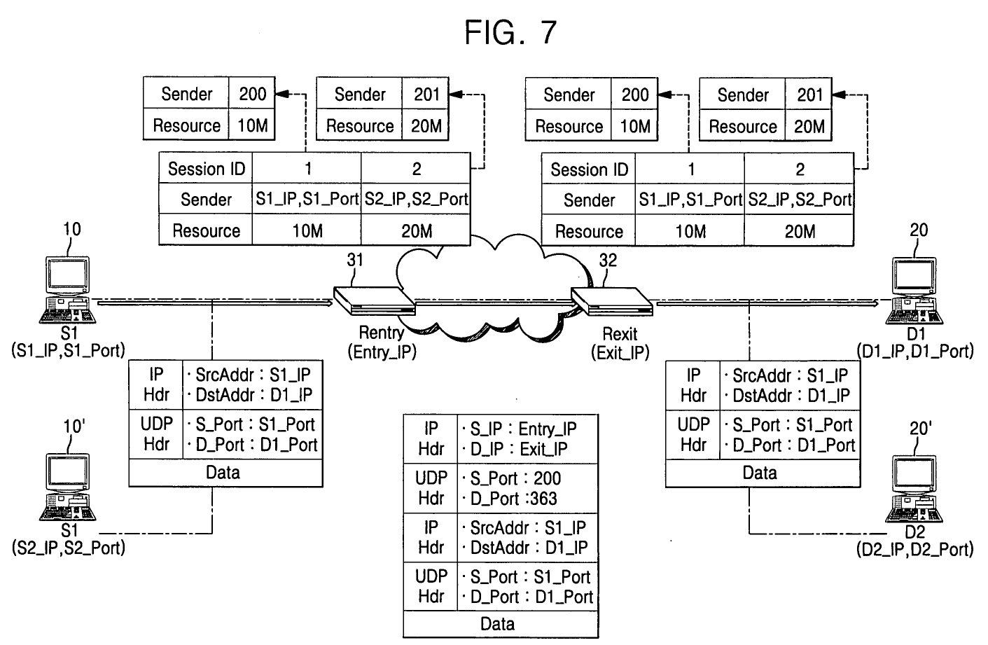

FIG. 7 is a diagram illustrating packet transmission through a plurality of tunnels in a typical IPv4/IPv6 hybrid network.

Referring to FIG. 7, a case wherein a first transmitting end 10 has reserved resources for transmitting a packet to a first receiving end 20 at 10 Mbps, and wherein a second receiving end 10′ has reserved resources for transmitting a packet to a second receiving end 20′ at 20 Mbps, will be described below.

Rentry 31 sets source port information for identifying sessions in a tunnel established for the first transmitting end 10 and the second transmitting end 10′ to “200” and “201,” respectively.

When receiving a packet from the first transmitting end 10, Rentry 31 encapsulates an IP header and a user datagram protocol (UDP) header of the packet for transmission to Rexit 32.

In the latter regard, destination port information of the encapsulated IP header and the UDP header of the packet are the same for all sessions, and source port information of the UDP header is set to a different value according to a session established between a transmitting end and a receiving end, and is then transmitted so that the RSVP-supportable node in the tunnel can identify each session based on the source port information of the received packet and can provide quality of service (QoS) required for each session.

Rexit 32 decapsulates the IP header and UDP header of the received packet for transmission to the receiving end 20.

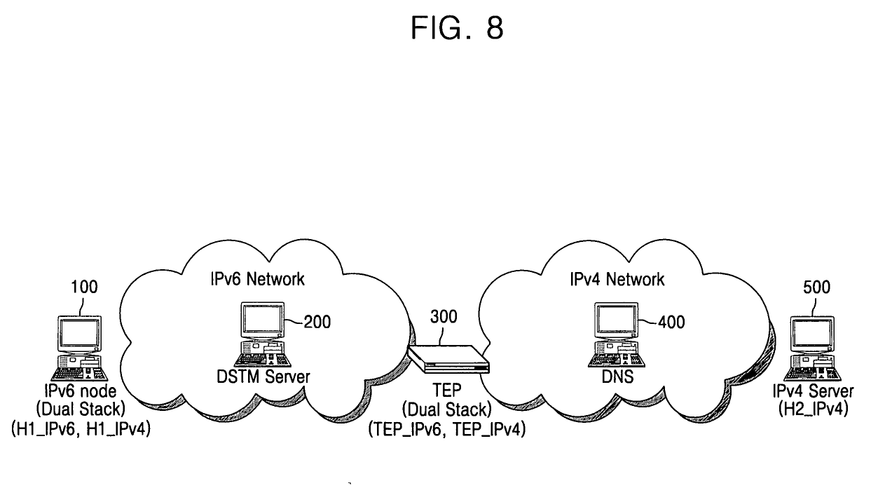

FIG. 8 is a diagram illustrating the configuration of a system for supporting RSVP in an IPv4/IPv6 hybrid network according to an exemplary embodiment of the present invention.

Referring to FIG. 8, the RSVP supporting system of the present invention comprises a dual stack host 100, a DSTM server 200, a DSTM terminal end point (TEP) 300, a domain name server (DNS) 400, and an IPv4-only server 500.

The dual stack host 100 is located in an IPv6 network. When the dual stack host 100 wants to receive multimedia traffic from the IPv4-only server 500 located in an IPv4 network and wants to ensure QoS for the traffic, the dual stack host 100 transmits a query message to the DNS 400 in order to obtain an IP address corresponding to the domain name of the server 500.

When receiving a response message including the IP address corresponding to the domain name of the IPv4-only server 500, the dual stack host 100 confirms that the IPv4-only server 500 is located in the IPv4 network.

Then, the dual stack host 100 transmits to the DSTM server 200 an address request message to obtain a temporary IPv4 address so as to connect to the IPv4-only server 500 using IPv4.

After transmitting the address request message, the dual stack host 100 is allocated one temporary global IPv4 address from the DSTM server 200, and is provided with IPv6 address information of the DSTM TEP 300.

The dual stack host 100 then transmits to the DSTM TEP 300 a message requesting to receive multimedia traffic based on QoS from the IPv4-only server 500.

In this regard, the message is transmitted to the DSTM TEP 300 by means of IPv4-in-IPv6 tunneling. More specifically, the message has an IPv4 header and is encapsulated again in an IPv6 packet.

When the dual stack host 100 receives a path message from the DSTM TEP 300 after transmitting the message requesting to receive multimedia traffic, the dual stack host 100 generates and transmits a Resv message using Ipv4-in-IPv6 tunneling in order to reserve resources in the IPv4 network.

In this regard, the Resv message transmitted to the DSTM TEP 300 has a structure which includes resource reservation request data (Resv data), an RSVP header (RSVP Hdr), an IPv4 header (IPv4 Hdr), and an IPv6 header (IPv6 Hdr).

In addition, the dual stack host 100 generates another Resv message, adds an IPv6 header to the message, sets a destination address to the DSTM TEP 300, and transmits the message using an IPv6 stack.

More specifically, the Resv message transmitted to the DSTM TEP 300 has a structure which includes path data, an RSVP header (RSVP Hdr), and an IPv6 header (IPv6 Hdr).

In this regard, the message is transmitted to the DSTM TEP 300 in the hop-by-hop manner, and reserves resources in each router existing in the IPv6 network during the transmission. When the packet arrives at the DSTM TEP 300, a QoS connection is established between the dual stack host 100 and the DSTM TEP 300.

When receiving the address request message transmitted from the dual stack host 100, the DSTM server 200 allocates the one temporary global IPv4 address to the dual stack host 100 and also provides the dual stack host 100 with the IPv6 address information of the DSTM TEP 300.

When the multimedia traffic reception request message is received from the dual stack host 100 by IPv4-in-IPv6 tunneling, the DSTM TEP 300 stores, in a table, mapping information between a source IPv6 address included in the received packet and an internal source IPv4 address, and then removes an IPv6 header and transmits an IPv4 packet having no IPv6 header to the IPv4-only server 500.

After transmitting the IPv4 packet having no IPv6 header to the IPv4-only server 500, the DSTM TEP 300 receives a path message defined in RSVP from the IPv4-only server 500.

The DSTM TEP 300 then extracts an IPv6 address from its own mapping table, the IPv6 address corresponding to an IPv4 address which is a destination address of the packet received from the IPv4-only server 500.

In other words, the DSTM TEP 300 encapsulates the path message into an IPv6 packet using the IPv6 address information extracted from the mapping table, and then transmits the IPv6 packet to the dual stack host using IPv4-in-IPv6 tunneling.

In this respect, the path message transmitted from the DSTM TEP 300 to the dual stack host 100 has a structure which includes path data, an RSVP header (RSVP Hdr), an IPv4 header (IPv4 Hdr), and an IPv6 header (IPv6 Hdr).

In order to reserve resources in the IPv6 network, the DSTM TEP 300 generates a path message of RSVP, adds an IPv6 header to the path message, and then transmits the path message to the dual stack host 100.

In this regard, the path message transmitted to the dual stack host 100 has a structure which includes path data, an RSVP header (RSVP Hdr), and an IPv6 header (IPv6 Hdr).

In addition, when receiving the Resv message through IPv4-in-IPv6 tunneling from the dual stack host 100, the DSTM TEP 300 removes the IPv6 header of the transmitted packet, and then transmits the IPv4 packet, including the Resv message, to the IPv4-only server 500. In this regard, the packet is transmitted in the hop-by-hop manner.

In other words, the packet transmitted to the IPv4-only server 500 has a structure which includes resource reservation request data (Resv data), an RSVP header (RSVP Hdr), and an IPv4 header (IPv4 Hdr).

In message transmission, resources are reserved in each router, and when the packet arrives at the IPv4-only server 500, a QoS connection is established between the DSTM TEP 300 and the IPv4-only server 500.

The DNS 400 confirms that the address corresponding to the domain name of the IPv4-only server 500 is an IPv4 address in response to the query message from the dual stack host 100, sets a type to “A,” and then transmits a response message, including the IPv4 address corresponding to the domain name of the IPv4-only server 500, to the dual stack host 100.

The IPv4-only server 500 is located in the IPv4 network for providing the dual stack host 100 with multimedia traffic. When a decapsulated QoS session request message is received from the DSTM TEP 300, the IPv4-only server 500 transmits the path message defined in RSVP to the dual stack host 100.

In other words, the path message transmitted by the IPv4-only server 500 is first transmitted to the DSTM TEP 300, and the path message has a structure which includes path data, an RSVP header (RSVP Hdr), and an IPv4 header (IPv4 Hdr).

In this respect, the destination address of an IPv4 packet included in the path message is an IPv4 address of the dual stack host 100, and the packet is transmitted to the DSTM TEP 300.

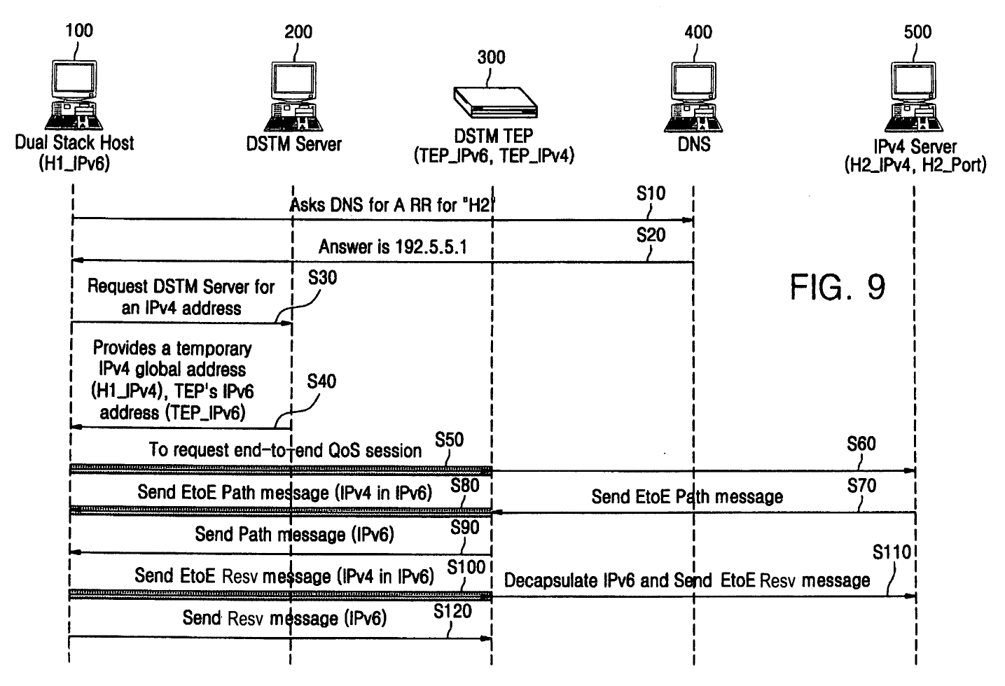

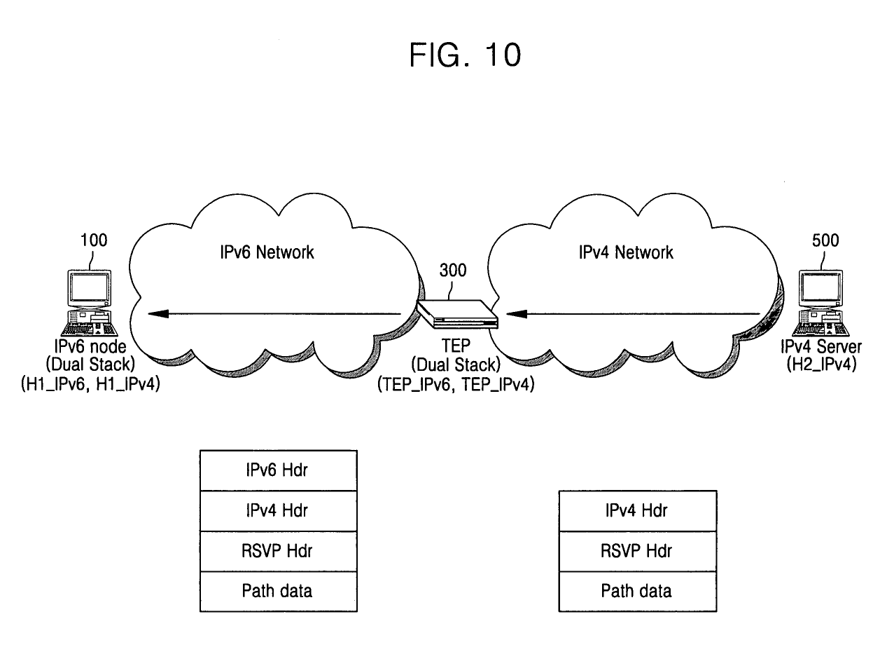

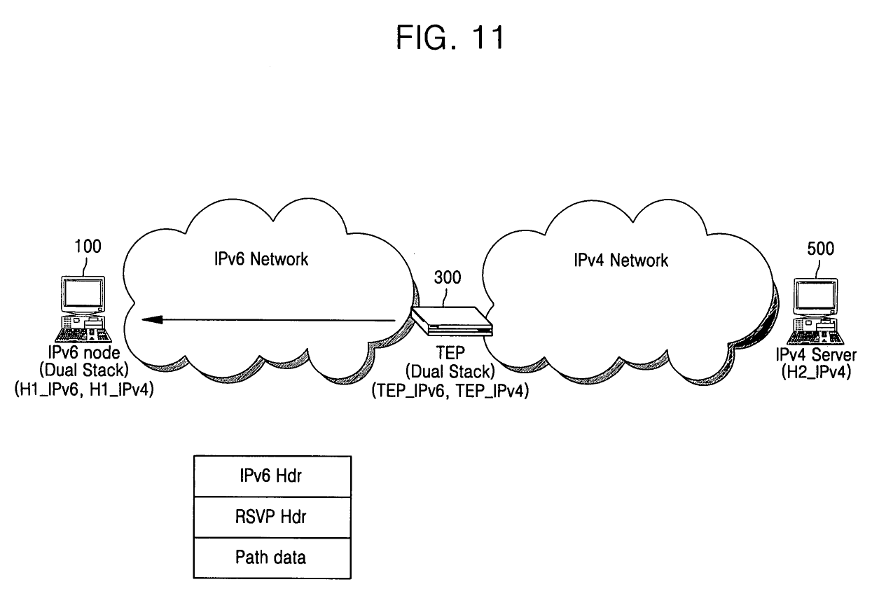

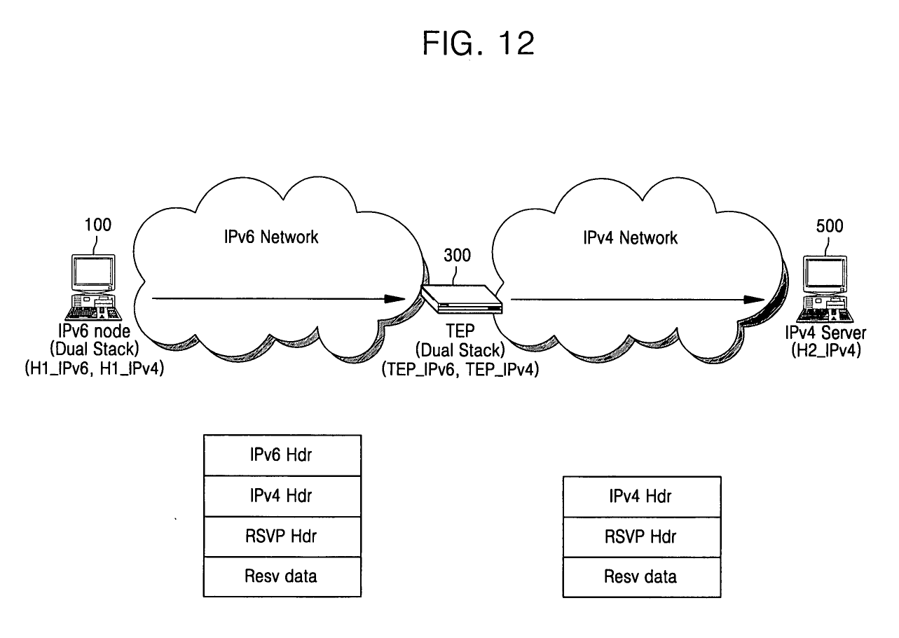

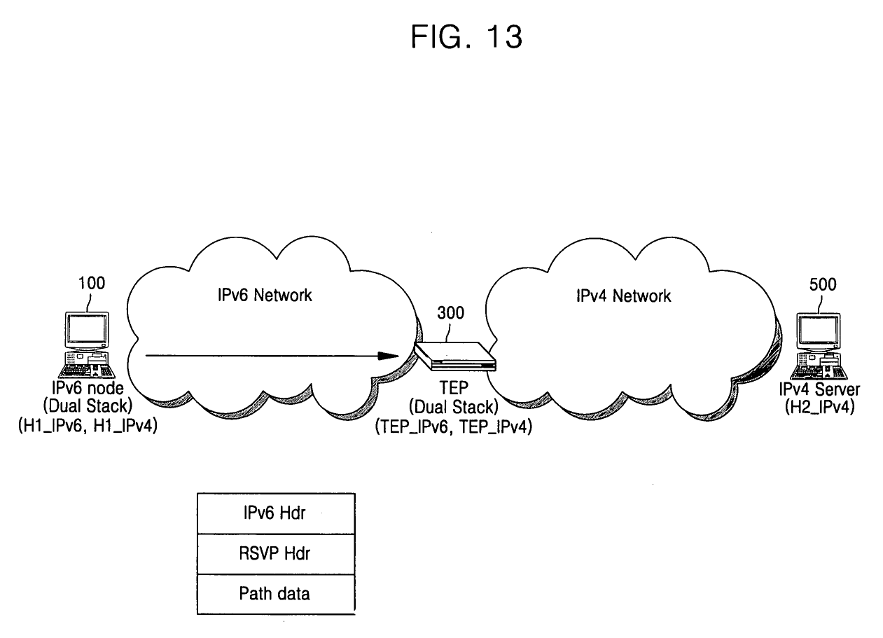

FIG. 9 is a diagram illustrating a method for supporting RSVP in an IPv4/IPv6 hybrid network according to an exemplary embodiment of the present invention, FIG. 10 is a diagram illustrating a process of transmitting an end-to-end path message in an IPv4/IPv6 hybrid network according to an exemplary embodiment of the present invention, FIG. 11 is a diagram illustrating a process of transmitting a message for reserving resources in an IPv6 network of an IPv4/IPv6 hybrid network according to an exemplary embodiment of the present invention, FIG. 12 is a diagram illustrating a process of reserving resources in an IPv4 network of an IPv4/IPv6 hybrid network according to an exemplary embodiment of the present invention, and FIG. 13 is a diagram illustrating a process of reserving resources in an IPv6 network of an IPv4/IPv6 hybrid network according to an exemplary embodiment of the present invention.

As illustrated in FIG. 9, when a dual stack host 100 located in the IPv6 network wants to receive multimedia traffic from an IPv4-only server 500 located in the IPv4 network and to ensure QoS for the traffic, the dual stack host 100 transmits a query message (Asks DNS for A RR for “H2”) to a DNS 400 in order to obtain an IP address corresponding to the domain name of the IPv4-only server 500 (S10).

In step S20, the DNS 400 confirms that the address corresponding to the domain name of the IPv4-only server 500 is an IPv4 address, sets a type to “A,” and then transmits a response message (Answer is 192.5.5.1) to the dual stack host 100.

In step S30, the dual stack host 100 receiving the response message from the DNS 400 confirms that the IPv4-only server 500 is located in the IPv4 network, and transmits an address request message (Request DSTM Server for an IPv4 address) to a DSTM server 200 in order to obtain one temporary IPv4 address to connect to the IPv4-only server 500 using an IPv4 protocol.

In step S40, the DSTM server 200 receiving the address request message from the dual stack host 100 allocates (Provides a temporary IPv4 global address (H1_IPv4), TEP's IPv6 address (TEP_IPv6)) one temporary global IPv4 address to the dual stack host 100.

In step S50, the dual stack host 100 transmits to the IPv4-only server 500 a message requesting to receive multimedia traffic based on QoS (To request end-to-end QoS session). The message is transmitted by means of IPv4-in-IPv6 tunneling. More specifically, the message has an IPv4 header and is again encapsulated into an IPv6 packet.

When the packet arrives at the DSTM TEP 300, the DSTM TEP 300 stores, in a table, mapping information between a source IPv6 address included in the packet and an internal source IPv4 address (cache association Hi_IPv6-H1_IPv4), removes an IPv6 header, and then transmits an IPv4 packet to the IPv4-only server 500 (S60).

In step S70, the IPv4-only server 500 sends a path message defined in RSVP to the dual stack host 100 (Send E to E Path message). In this regard, the destination address of an IPv4 packet including the message is an IPv4 address of the dual stack host 100, and the packet is transmitted to the DSTM TEP 300.

In other words, as illustrated in FIG. 10, the path message transmitted from the IPv4-only server 500 is transmitted to the DSTM TEP 300 first. In this regard, the path message has a structure which includes path data (Path data), an RSVP header (RSVP Hdr), and an IPv4 header (IPv4 Hdr).

Furthermore, the destination address of the IPv4 packet included in the path message is the IPv4 address of the dual stack host 100, and the packet is transmitted to the DSTM TEP 300.

In step S80, the DSTM TEP 300 extracts an IPv6 address corresponding to the IPv4 address, which is the destination address of the packet, from its own mapping table, encapsulates the IPv4 packet in an IPv6 packet using the information, and then sends the IPv6 packet to the dual stack host 100 using IPv4-in-IPv6 tunneling (Send E to E Path message (IPv4-in-IPv6)).

More specifically, as illustrated in FIG. 10, the DSTM TEP 300 extracts the corresponding IPv6 address from its own mapping table using the IPv4 address which is the destination address of the packet received from the IPv4-only server 500.

After encapsulating the IPv4 packet in the IPv6 packet using the IPv6 address information extracted from the mapping table, the DSTM TEP 300 transmits the IPv6 packet to the dual stack host 100 using IPv4-in-IPv6 tunneling.

In this regard, the path message transmitted from the DSTM TEP 300 to the dual stack host 100 has a structure which includes path data (Path data), an RSVP header (RSVP Hdr), an IPv4 header (IPv4 Hdr), and an IPv6 header (IPv6 Hdr).

In step S90, in order to reserve resources in the IPv6 network, the DSTM TEP 300 generates a path message defined in RSVP, adds an IPv6 header to the path message, and transmits the path message to the dual stack host 100 (Send Path message (IPv6)).

In other words, as illustrated in FIG. 1, in order to reserve resources in the IPv6 network, the DSTM TEP 300 generates the path message defined in RSVP, adds an IPv6 header to the path message, and transmits the path message to the dual stack host 100.

In this regard, the path message transmitted to the dual stack host 100 has a structure which includes path data (Path data), an RSVP header (RSVP Hdr), and an IPv6 header (IPv6 Hdr).

In step S100, in order to reserve resources in the IPv4 network, the dual stack host 100 receiving the path message generates and transmits (Send EtoE Resv message (IPv4 in IPv6)) a Resv message using IPv4-in-IPv6 tunneling.

In other words, as illustrated in FIG. 12, when the dual stack host 100 receives the path message from the DSTM TEP 300 after transmitting the message requesting to receive multimedia traffic, the dual stack host 100 generates and transmits the Resv message using IPv4-in-IPv6 tunneling in order to reserve resources in the IPv4 network.

In this regard, the Resv message transmitted to the DSTM TEP 300 has a structure which includes resource reservation request data (Resv data), an RSVP header (RESVP Hdr), an IPv4 header (IPv4 Hdr), and an IPv6 header (IPv6 Hdr).

When the packet is transmitted to the DSTM TEP 300, the DSTM TEP 300 removes the IPv6 header and then transmits an IPv4 packet including the Resv message to the IPv4-only server 500 (Decapsulate IPv6 and Send E to E Resv message), in step S110. Here, the packet is transmitted to the server in the hop-by-hop manner.

As illustrated in FIG. 12, the packet transmitted to the IPv4-only server 500 has a structure which includes resource reservation request data (Resv data), an RSVP header (RSVP Hdr), and an IPv4 header (IPv4 Hdr).

In message transmission, resources are reserved in each router. When the packet arrives at the IPv4-only server 500, a QoS connection is established between the DSTM TEP 300 and the IPv4-only server 500. With this alone, however, an end-to-end QoS connection is not established.

In order to establish an end-to-end QoS connection, resource reservation must be made in the IPv6 network. To this end, in step S120, the dual stack host 100 generates another Resv message, adds an IPv6 header to the message, sets a destination address to the DSTM TEP 300, and then transmits the message using an IPv6 stack (Sends Resv message (IPv6)).

As illustrated in FIG. 13, the Resv message transmitted to the DSTM TEP 300 has a structure which includes path data (Path data), an RSVP header (RSVP Hdr), and an IPv6 header (IPv6 Hdr).

In this latter regard, the message is transmitted to the DSTM TEP 300 in the hop-by-hop manner, and resources are reserved in each router in message transmission. When the packet arrives at the DSTM TEP 300, a QoS connection is established between the dual stack host 100 and the DSTM TEP 300.

As a result, when the QoS connections are established between the dual stack host 100 and the DSTM TEP 300, and between the DSTM TEP 300 and the IPv4-only server 500, the end-to-end QoS connection is established.

According to the present invention, when a server for multimedia traffic is located in an IPv4 network of an IPv4/IPv6 hybrid network in which IPv4 and IPv6 networks coexist, traffic transmitted from the server to a dual stack host located in the IPv6 network can be assured QoS. Therefore, it is possible to ensure QoS through an end-to-end RSVP connection.

While the present invention has been described with reference to exemplary embodiments thereof, it will be understood by those skilled in the art that various changes in form and detail may be made therein without departing from the scope of the present invention as defined by the following claims

What is claimed is:

1. A method for supporting resource reservation protocol (RSVP) in an Internet protocol version 4 (IPv4)/Internet protocol version 6 (IPv6) hybrid network in which an IPv4 network and an IPv6 network are combined by a dual stack transition mechanism terminal end point (DSTM TEP), the method comprising the steps of:
(a) transmitting an end-to-end quality of service (QoS) session establishment request message from a dual stack host in the IPv6 network to an IPv4 server through the DSTM TEP;
(b) transmitting an end-to-end path message from the IPv4 server to the dual stack host through the DSTM TEP;
(c) transmitting a path message for reserving resources in the IPv6 network from the DSTM TEP to the dual stack host;
(d) transmitting an end-to-end resource reservation request message from the dual stack host to the IPv4 server through the DSTM TEP, and making a resource reservation in the IPv4 network; and
(e) transmitting a resource reservation request message from the dual stack host to the DSTM TEP and making a resource reservation in the IPv6 network.

2. The method of claim 1, wherein in step (a), the end-to-end QoS session establishment request message has an IPv4 header, is encapsulated in an IPv6 packet, and is transmitted to the DSTM TEP by means of IPv4-in-IPv6 tunneling.

3. The method of claim 2, wherein the DSTM TEP stores, in a table, mapping information between a source IPv6 address included in the message transmitted from the dual stack host and an internal source IPv4 address, and then transmits to the IPv4 server an IPv4 packet from which an IPv6 header is removed.

4. The method of claim 1, wherein in step (b), the end-to-end path message transmitted to the DSTM TEP has a structure which includes path data, an RSVP header, and an IPv4 header.

5. The method of claim 4, wherein a destination address of an IPv4 packet which includes the end-to-end path message is an IPv4 address of the dual stack host.

6. The method of claim 5, wherein the DSTM TEP extracts, from a mapping table, an IPv6 address mapped to the IPv4 address corresponding to the destination address of the IPv4 packet, encapsulates the IPv6 address in an IPv6 packet, and then transmits the IPv6 packet to the dual stack host using IPv4-in-IPv6 tunneling.

7. The method of claim 6, wherein the IPv6 packet transmitted using IPv4-in-IPv6 tunneling has a structure which includes path data, an RSVP header, an IPv4 header, and an IPv6 header.

8. The method of claim 1, wherein in step (c), the path message has a structure which includes path data, an RSVP header, and an IPv6 header.

9. The method of claim 1, wherein in step (d), the end-to-end resource reservation request message has an IPv4 header, is encapsulated in an IPv6 packet, and is transmitted to the DSTM TEP by means of IPv4-in-IPv6 tunneling.

10. The method of claim 9, wherein the resource reservation request message transmitted to the DSTM TEP has a structure which includes resource reservation request data, an RSVP header, an IPv4 header, and an IPv6 header.

11. The method of claim 10, wherein the DSTM TEP transmits to the IPv4 server an IPv4 packet which is the resource reservation request message from which an IPv6 header is removed.

12. The method of claim 11, wherein the IPv4 packet transmitted to the IPv4 server makes a resource reservation in each router while being transmitted in a hop-by-hop manner.

13. The method of claim 1, wherein in the step of transmitting, at the dual stack host, a resource reservation request message to the DSTM TEP and making a resource reservation in the IPv6 network, the resource reservation request message has a structure including path data, an RSVP header, and an IPv6 header.

14. The method of claim 13, wherein the resource reservation request message transmitted to the DSTM TEP makes a resource reservation in each of a plurality of routers while being transmitted in a hop-by-hop manner.

15. A system for supporting resource reservation protocol (RSVP) in an IPv4/IPv6 hybrid network in which an IPv4 network and an IPv6 network are combined by a dual stack transition mechanism terminal end point (DSTM TEP), the system comprising:
a dual stack host for transmitting an end-to-end resource reservation request message to an IPv4 server through the DSTM TEP so as to make a resource reservation in the IPv4 network upon receipt of an end-to-end path message from the IPv4 server through the DSTM TEP, and transmitting a resource reservation request message to the DSTM TEP so as to make a resource reservation in the IPv6 network.

16. The system of claim 15, wherein the end-to-end path message transmitted to the DSTM TEP has a structure which includes path data, an RSVP header, and an IPv4 header.

17. The system of claim 16, wherein a destination address of an IPv4 packet which includes the end-to-end path message is an IPv4 address of the dual stack host.

18. The system of claim 17, wherein the DSTM TEP extracts, from a mapping table, an IPv6 address mapped to the IPv4 address corresponding to the destination address of the IPv4 packet, encapsulates the IPv6 address in an IPv6 packet, and then transmits the IPv6 packet to the dual stack host using IPv4-in-IPv6 tunneling.

19. The system of claim 18, wherein the IPv6 packet transmitted using IPv4-in-IPv6 tunneling has a structure which includes path data, an RSVP header, an IPv4 header, and an IPv6 header.

20. The system of claim 15, wherein the DSTM TEP transmits a path message for reserving resources in the IPv6 network to the dual stack host.

21. The system of claim 20, wherein the path message for reserving resources in the IPv6 network has a structure which includes path data, an RSVP header, and an IPv6 header.

22. The system of claim 15, wherein the end-to-end resource reservation request message transmitted to the DSTM TEP has an IPv4 header, is encapsulated in an IPv6 packet, and is transmitted to the DSTM TEP by means of IPv4-in-IPv6 tunneling.

23. The system of claim 22, wherein the end-to-end resource reservation request message transmitted to the DSTM TEP has a structure which includes resource reservation request data, an RSVP header, an IPv4 header, and an IPv6 header.

24. The system of claim 23, wherein the DSTM TEP transmits, to the IPv4 server, an IPv4 packet which is the end-to-end resource reservation request message from which an IPv6 header is removed, the end-to-end resource reservation message being transmitted from the dual stack host.

25. The system of claim 24, wherein the IPv4 packet transmitted to the IPv4 server makes a resource reservation in each of a plurality of routers while being transmitted in a hop-by-hop manner.

26. The system of claim 15, wherein the end-to-end resource reservation request message transmitted to the DSTM TEP has a structure which includes path data, an RSVP header, and an IPv6 header.

27. The system of claim 26, wherein the end-to-end resource reservation request message transmitted to the DSTM TEP makes a resource reservation in each of a plurality of routers while being transmitted in a hop-by-hop manner.
